(19) United States
(12) Patent Application Publication
(10) Pub. No.: US 2012/0181349 A1
(43) Pub. Date: Jul. 19, 2012

(54) Device for dispensing a volatile substance, processes for manufacturing such a device and its use

(75) Inventors: Helge Adleff, Berlin (DE); Thilo Guschauski, Berlin (DE)

(73) Assignee: ACUROS GMBH, Berlin (DE)

(21) Appl. No.: 13/354,054

(22) Filed: Jan. 19, 2012

(30) Foreign Application Priority Data
Jan. 19, 2011 (DE) 102011000223.5

(51) Int. Cl. A61L 9/04

(52) U.S. Cl. 239/34

(57) ABSTRACT

Device for dispensing a volatile active substance, comprising a container (3) that is at least partially restricted by a transpiration layer (2). The container (3) is at least partially filled with a liquid mixture comprising at least one volatile liquid substance and an auxiliary liquid. The liquid mixture of the liquid active substance and the auxiliary liquid is capable of forming an azeotrope. The transpiration layer (2) is impermeable for the liquid phase of the liquid mixture, but permeable to its gaseous phase, however.

### CROSS-REFERENCE TO RELATED APPLICATIONS

This patent application claims priority of the German patent application No. 102011000223.5 filed on Jan. 19, 2011, which is incorporated herein by reference.

### BRIEF SUMMARY OF THE INVENTION

The present invention relates to a device such as a dispenser or a dosage form for controlled release of a volatile substance, its production and use for dispensing a volatile substance.

### BRIEF DESCRIPTION OF THE SEVERAL VIEWS OF THE DRAWINGS

Hereinafter, the present invention is exemplified with reference to figures. It shows:

FIG. 1 the construction of a closed and sealed dispensing device;

FIG. 2 the construction of a dispensing device according to an exemplary embodiment during opening and when open;

FIG. 3 the construction of a dispensing device according to another exemplary embodiment;

FIG. 4 the cross section of another embodiment of a dispensing device;

FIG. 5 the construction of a dispensing device according to FIG. 4, located in an envelope with an opening;

FIG. 6 the temporal course of release of formic acid by a dispensing device in different operating states according to FIG. 1 and FIG. 2.

### BACKGROUND OF THE INVENTION

Regarding the respective application the dispensing of volatile substances is subject of such diverse cases like repelling or attracting pests, aromatization and deodorization, decontamination, signaling, sterilization and human or veterinary treatments. Controlled release is of particular interest, i.e. controlling the release kinetics of the agent over long periods.

The dispensing of organic acids is of particular interest in terms of their usefulness as a drug.

For example, the dispensing of formic acid in beehives is a known method of controlling varroa mites in infested hives. Varroa mites are parasites of the honey bee and cause increasingly substantial losses of bee colonies around the world. A known method for the treatment of Varroa is to sustain a specific vapor pressure of formic acid in the hive for a period of several days to several weeks, whereby the concentration of formic acid is letal to the mites and for the majority of the bees is however only subletal.

Different methods and devices for the dispensing of formic acid are known, all of which involve the risk of direct contact with liquid formic acid.

DD 292141 and WO 97/32470 disclose methods and devices for evaporation of fluids, especially formic acid, which use the capillary flow in an evaporation means. Devices described herein comprise a container and an evaporation chamber. DE 3427330 discloses a device for Varroa treatment comprising a softboard soaked with concentrated formic acid and covered by a plastic bag with 5 to 15 holes on every side. WO 94/19043 describes a volatile liquid dispenser for administering medication through a film impermeable to liquids.

### DETAILED DESCRIPTION OF THE INVENTION

According to claim 1 a device, e.g. a dispensing device, for controlled release or dispensing of a volatile agent is provided. A process for producing a device is provided according to claim 8 or 10. Furthermore, applications of the device to release or dispense the volatile substance are proposed. Further advantageous embodiments, details and features of the present invention will become apparent from the subclaims, the description, the embodiments and the accompanying figures.

According to an exemplary embodiment of the present invention it is proposed to dispense a volatile substance to the environment by means of transpiration through a barrier layer, the timely concentration trend being predefined by the composition of the liquid mixture. Thereby, it is never any direct contact between the liquid phase of volatile substance and the environment.

Different from previously known applications of a barrier layer, the mixture of the liquid agent and an auxiliary liquid is subject to change during the dispensing process. The changing composition can be used to i.e. incipiently control the trend of the amount of gaseous agent dispensed to the environment.

Thus, in a defined area with known exchange rates, i.e. values of the inflowing and outflowing volumes per time, time of reaching a predefined average concentration of the active ingredient can be adjusted. Similarly, a temporal profile of the average concentration of the active ingredient can be adjusted, for example in the form of a biphasic release profile.

First, the term azeotrope which is used for a detailed description of the invention shall be explained. As used below, an azeotrope should be understood to be a mixture of at least two liquids where the composition of the vapor of the mixture is the same (ex. concentration ratio) as of the liquid phase of the mixture.

According to the ratio of boiling temperatures of the mixture and the individual liquids positive and negative azeotropes can be distinguished. With positive azeotropes, the mixture has a lower boiling point than the respective individual liquids. With negative azeotropes, the boiling point of the mixture is higher than the boiling point of the respective individual liquids. At the so-called azeotropic point, the vapor phase and the solution of the azeotropic mixture are of the same composition and the concentration of both phases can not be changed by distillation.

According to various embodiments, a device or dispensing device 10, such as a dispenser for dispensing or supply of a liquid substance (or a volatile liquid substance) comprises a container 3, which forms a receptacle for the liquid volatile agent 4, and a transpiration surface 2 which is permeable to the gaseous volatile substance but repellent and impermeable to the active substance in its liquid form. The container 3 of the operational dispenser contains a liquid mixture of the active ingredient with an auxiliary liquid. As an auxiliary liquid substances may be used which form an azeotrope with the active ingredient.

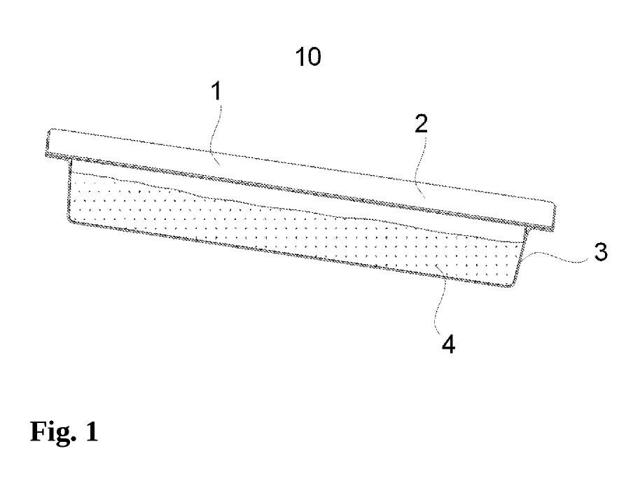

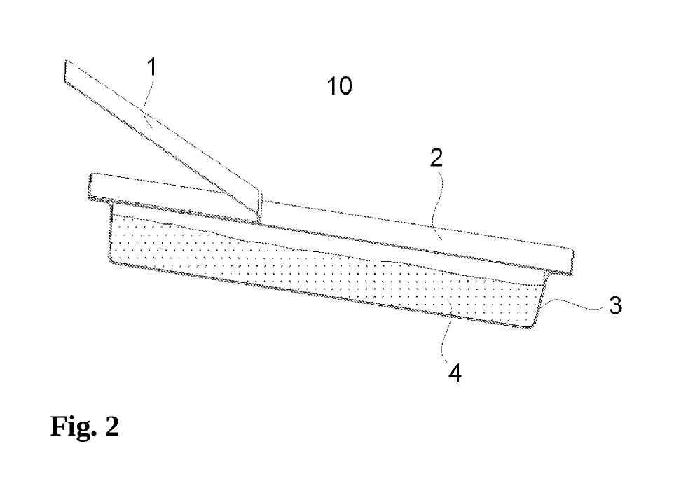

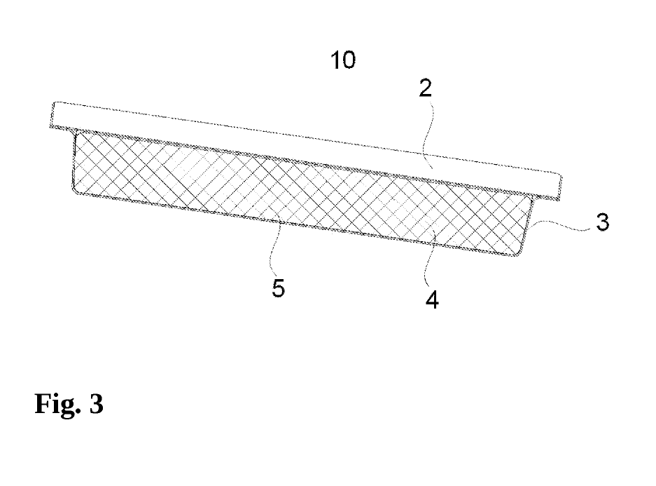

As shown in FIGS. 1 to 5, the exemplary dispensing device 10 comprises a container 3 for the volatile liquid agent, which is referred to below as the substance. The device 10 is bordered at least in part by a porous barrier used as a transpiration layer 2, which is repellent to the liquid volatile agent 4 but permeable to its gaseous form, however.

The transpiration layer 2 may be a porous layer, a membrane, a foil or film. For example, the transpiration layer may be constructed in the form of a suitable barrier. Examples include films made of a hydrophobic or oleophobic material. For example, the material of the transpiration layer may be polyethylene, polypropylene, polytetrafluoroethylene, or another material having a low surface energy. Similarly, the transpiration layer can be formed of a metal layer, a ceramic layer, a nuclear track membrane or a paper. Alternatively, the material of the porous barrier used as a transpiration layer 2 can be pre-treated chemically or physically so that it exhibits hydrophobic and oleophobic properties. The pore diameter of 2 transpiration layer be for example between less than 10 nanometers and more than 10 micrometers. Thus, layers, membranes, foils, or films suitable to be used as a transpiration layer 2 constitute ultra- and/or nano- and/or microporous layers, membranes, foils, or films.

The transpiration layer 2 can at least temporarily, and especially prior to use for gaseous release of liquid volatile active substances be covered by an impermeable barrier 1, for example in the form of a barrier layer, which is impermeable to the volatile agent 4 in the liquid and gaseous condition. Also the impermeable barrier layer may consist of a hydrophobic or oleophobic or of a pre-treated hydrophobic or oleophobic material. Also, the impermeable barrier can constitute a thin metal foil such as aluminum foil or a thin polymer film with a metal layer.

The impermeable barrier 1 may be arranged so that it can be removed or otherwise disseminated in whole or in part prior to use of the device 10 so to start the release process by transpiration layer 2.

The container 3 can be filled partially or completely with a wettable porous material 5 in its interior, which acts as a storage matrix for the even distribution of liquid volatile substance 4 in the container 3 and to support a steady transpiration through the transpiration layer 2. The container 3 and/or the wettable porous material 5 form a drug reservoir. In addition, the wettable porous material 5 functioning as a storage matrix, or the drug reservoir formed by it, can support the transpiration layer mechanically. A similar effect can be achieved by a wettable porous layer 2a, which contacts the transpiration layer (porous barrier) 2.

The wettable porous material 5 functioning as a storage matrix may consist for example of cellulose, viscose, polyester, polyamide, or another material which is wettable or is treated to be wettable by the volatile liquid substance 4 and the auxiliary liquid.

In an exemplary embodiment shown the in the FIGS. 1-3, the device 10 consists of one single material. For example, a polypropylene cup forms a container 3 and represents the external form of the dispenser. A porous film of polypropylene forms the porous barrier as a transpiration layer 2 and is sealed to the cup, thereby closing the cup 2. A non-porous polypropylene film forms an impermeable barrier layer 1 and is sealed on top of the porous foil on the edge of the cup forming the container. To start the release process, it is removed in whole or in part.

According to an exemplary embodiment, an impermeable barrier layer 1 has one or several openings or holes arranged adjacent to the transpiration layer 2, which are initially closed by a segment or several independent segments of an impermeable barrier. By the number of that one or several openings simultaneously uncovered by removal of segments, the release kinetics and the running time of the dispensing device can be controlled and adjusted to the respective room volume or room size or to the specific application.

Alternatively, all plastic parts of the apparatus described can consist of, for example, polytetrafluoroethylene, polyethylene or other polyolefin, for example, a cyclic olefin polymer or cyclic olefin copolymer.

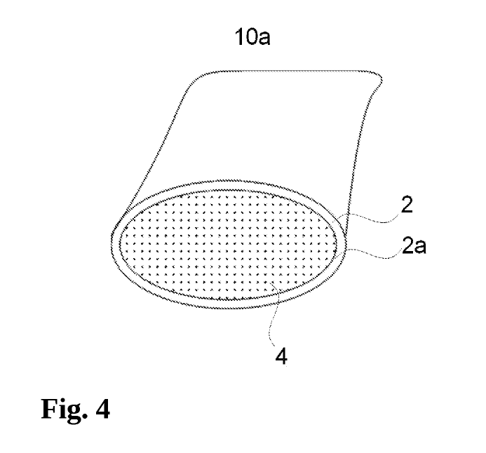

A further exemplary embodiment 10a of device 10 is a pouch or a tube closed at both ends, made from one or more flexible foils or foil laminate. Here, the bag defines a receiving space. Such a device (dispenser) 10a is shown in FIG. 4. At least one of the foils constitutes an porous barrier functioning as a transpiration layer 2 in operating condition. The above-mentioned function of a mechanical support in the embodiment of the dispenser 10a can be achieved by a wettable porous layer 2a, which also acts as a storage matrix and contacts the porous barrier 2. The bag is filled with a mixture of volatile liquid substance 4 and an auxiliary liquid and transpiration takes place on that part of the surface of the bag, which is formed by the transpiration layer 2. Alternatively, the transpiration takes place on the entire surface of the bag when it is completely formed from an appropriate porous film. Porous foils suitable for use as transpiration layers are, for example, porous membranes made of polypropylene, polyethylene, polytetrafluoroethylene, expanded polytetrafluoroethylene, polyethersulfone, or another substance that has been coated with any of those substances, or other membranes such as cellulose or regenerated cellulose, which have received hydrophobic or oleophobic properties by coating.

A device in the form of a tubular dispenser 10b can be packed in a suitable envelope which serves as a container 3 up to the moment of beginning of operation, the envelope comprising a predetermined break zone (tear). In an example, such an envelope represents a flexible unpermeable barrier layer 1. Thus, for example a tube made from an impermeable material can be considered as an envelope which is closed at its ends 6 by sealing over its entire width 8.

Alternatively, such a tubing or a tube closed at both ends may have one or more windows 9 or perforations. By removing a impermeable film which is laminated on or over a window or perforations, this can be opened. This offers the advantage that a removal of the dispenser 10b from the container 3 serving as envelope is not required for the purpose of drug release.

The storage matrix of the dispenser 10b is soaked with the mixture containing the active ingredient 4 and is enclosed inside the container 3, for example, by a porous barrier which is completely suitable as a transpiration layer 2.

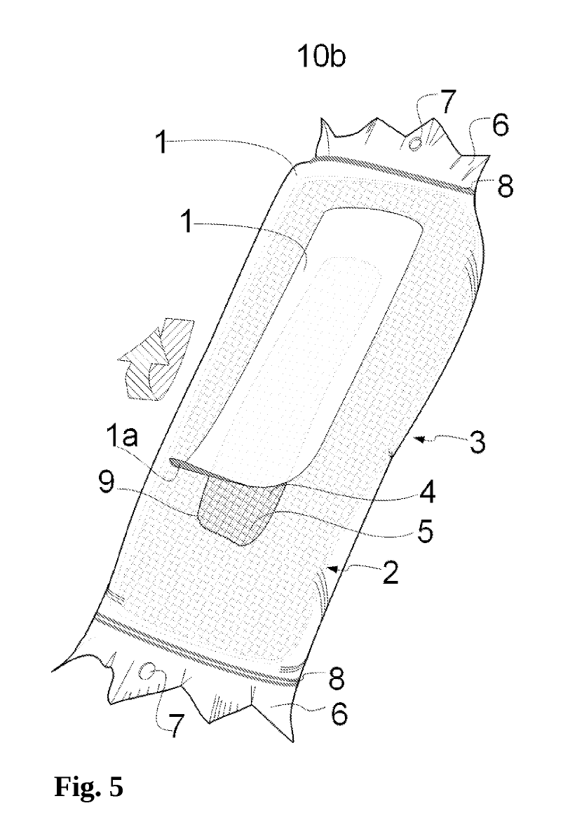

FIG. 5 shows a perspective view of an exemplary embodiment of such a dispenser 10b. The segment of an impermeable foil 1 (see hatched arrow), for example in the form of a strap-deductible segment here, can be attached by means of lamination or with an adhesive on its underside 1a on a film tube serving as a container, so that the window 9 is closed initially. In addition, the container 3 may be provided with small openings 7 at the outer edge of one or both of its ends to allow attachment of suitable fastening means.

A described dispenser may firstly be offset already in production to an operational state, i.e. contain the mixture of liquid volatile agent and auxiliary liquid in a storage matrix under a porous barrier suitable as transpiration layer, which in turn is delimited from the outer world by an impermeable barrier layer. Secondly, it is also possible to provide a wettable porous material 5 suitable as a storage matrix, which is non-enveloped or only partially enveloped by an impermeable barrier 1, which is then completely or at least partially enclosed by the transpiration layer which is suitable as porous barrier 2. In case of partial coverage adjacent edges of the impermeable barrier 1 and the porous membrane suitable as transpiration layer 2 are firmly connected. The so coated storage matrix does not contain the active ingredient initially.

The filling of such a dispenser, initially containing no active ingredient can eg take place by means of injection of a mixture prepared on site by a portion of the exposed transpiration layer into the storage matrix. The injection opening in the transpiration layer caused thereby can be sealed with a segment of an impermeable barrier coated with glue or with a drop of glue.

This offers the advantage of being able to adjust drug release kinetics to the respective applications, e.g. In the context of release cycles comprising different dosing schemes.

Thus, the described mixture of a volatile active substance with an auxiliary liquid forming an azeotrope with the active substance is the formulation of the drug. A dispenser filled with the formulated drug in turn represents a dosage form for systemic and/or inhaled administration of a drug.

According to an embodiment, the initial concentration of the active substance in the liquid mixture is greater than the concentration of the drug at the azeotropic point of the liquid mixture. According to another embodiment, the initial concentration of the active substance in the liquid mixture is lower than the concentration of the drug at the azeotropic point of the liquid mixture. According to another embodiment, the initial concentration of the active substance in the liquid mixture corresponds to the concentration of drug at the azeotropic point of the liquid mixture. The initial concentration here means the concentration when filling the device.

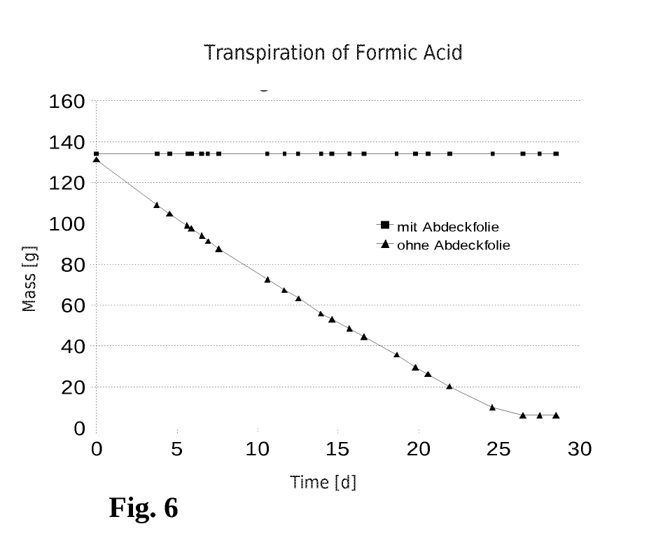

As indicated in the horizontal curve of the measured values in FIG. 6, a dispenser of the type described above can be kept without loss of the encapsulated volatile active substance 4 when completely enveloped by an impermeable membrane (The mass of the liquid mixture remains constant over time). Dispensing of volatile substance 4 occurs only after the impermeable cover film (barrier layer) was withdrawn. As indicated in the profile of the second, falling, curve in FIG. 6, the transpiration rate is almost constant without the impermeable film (The mass of the liquid mixture is reduced at a constant rate) until the volatile substance 4 is completely dispensed.

Depending on the composition of the mixture of at least two azeotrope-forming liquids (the active substance and the auxiliary liquid) in the dispenser 10, 10a, 10b, or in its wettable porous material, or the storage matrix 5, different dispensing profiles an active substance can be achieved. For example, the composition of a mixture of the liquid active ingredient with an auxiliary liquid is used to control the release of the first phase of gaseous active substance discharged into the environment. In particular, the initial concentration the active substance in the gas phase is determined.

The specified concentration of the active ingredient in the mixture determines the temporal course of release, i.e. the release kinetics. Thus, the choice of the auxiliary liquid that can form an azeotrope with the agent and the chosen concentration of the active ingredient in the mixture, relative to the azeotropic point of the mixture, determine the first phase of the release profile until reaching the azeotropic point. When the concentration of active substance reaches the azeotropic point, no further change occurs in the concentration of active substance (and the auxiliary liquid) in the subsequent release or during the second phase of the release.

Thus, the azeotropic concentration of active ingredient can be achieved starting from higher concentrations as well as from concentration values below the azeotropic point. It is also possible to set the concentration of the active substance in the gasuous phase from the beginning and during the entire release duration to such a value that is close or equal to that at the azeotropic point of the mixture of active ingredient and auxiliary liquid.

### Example 1

In one example, 77.5% formic acid, used in accordance with the azeotropic point of aqueous formic acid, is used as a volatile liquid substance 4 in a dispenser 10. When the impermeable barrier layer 1 is removed and gaseous formic acid can be released through the transpiration layer 2 which is impermeable to liquids, the concentration of formic acid remains the same during the entire dispensing process. Therefore, the dispensing rate of formic acid does not change. The release kinetics achieved is due to the fact that formic acid forms a negative azeotrope with water.

### Example 2

In another example, 85% formic acid is used as a volatile liquid substance 4 in a dispenser 10. In the gaseous phase, the concentration of formic acid is above 85% in the beginning of the dispensing process. Hence, the concentration in the liquid pase is slowly decreasing until it equals the azeotropic point at 77.5%. Then the concentration of formic acid in the liquid mixture (in the storage matrix) will not change any more. The concentration of formic acid in the gas phase follows this concentration gradient. Accordingly, the dispensing rate of formic acid is highest at the beginning, decreases gradually and approaches a constant value which corresponds to the concentration in the azeotropic point.

### Example 3

In another example, 60% formic acid is used as a volatile liquid substance 4 in a dispenser 10. In the gaseous phase, the concentration of formic acid is below 60% in the beginning of the dispensing process. Hence, the concentration in the liquid pase is slowly increasing until it equals the azeotropic point at 77.5% and now does not change any further. The concentration of formic acid in the gas phase follows this concentration gradient. Accordingly, the dispensing rate of formic acid is lowest at the beginning, increases gradually and approaches a constant value.

### Example 4

A mixture of chloroform and methanol forms a positive azeotrope, because the vapor pressure of the solution is above the theoretical value according to the Raoult's law for ideal mixtures. The azeotropic point for this mixture is at 87% chloroform and 13% methanol.

In one example, a mixture of 87% chloroform and 13% methanol in accordance with the azeotropic point of the mixture, is used as a volatile liquid substance 4 in a dispenser 10. The composition of the gas phase does not differ from the liquid pahse throughout the application. The dispensing rate of chloroform and methanol, respectively, is consistent.

### Example 5

In another example, a mixture of 80% chloroform and 20% methanol is used. Since this is a positive azeotropic mixture, the composition of the gas phase is closer to the azeotropic point than the composition of the liquid phase. With continuing evaporation of the mixture, the concentration of the liquid and the gas phase is thus increasingly turned away from the azeotropic point. In the present example, the concentration of chloroform in the vapor is thus higher than 80% in the beginning and decreases gradually until the mixture is completely vaporized, thus following the concentration of the liquid phase. The dispensing rate of chloroform decreases therefore continuously during the application.

For the component of the positive azeotropic mixture, the concentration of which is above the azeotropic point, methanol in the illustrated example, the reverse applies.

The same applies to all liquid volatile substances which may form a positive azeotrope.

### Example 6

In another embodiment, the volatile substance is a pheromone, or a compound with phoromone like activity, or represents a mixture thereof. For example, pheromone traps equipped with a dispenser 10, 10a, 10b are used to trap insect pests. By targeted choice of the pheromone, the respective kinetics of release and site of the dispenser, it is possible to repell or to confuse insect pests. An application designed according to this example of the dispenser can be used, for example, in fruit, vegetables or ornamental plants. For example, infection densities in open or closed systems can be determined. Likewise pests in storage or utility rooms can be controlled or pest infestation can be monitored.

### Example 7

According to a further use of a dispenser of the type described, the atmosphere of storage rooms, warehouses, sales rooms, living rooms, bedrooms and bathrooms patient rooms can be enriched with active ingredients, for example, to control pests, for deodorization, to increase well-being or for relief from respiratory ailments. For example, the interior of a motor vehicle or an aircraft can be deodorised, be flavored or enriched with substances to increase well-being.

### Example 8

According to another embodiment, the volatile substance contains formic acid and is used for the treatment of the Varroa in beehives. An example of formic acid concentration is between 40% and 98%, in another exemplary embodiment, the concentration is 60%, in another exemplary embodiment it is 77.5% and in a further embodiment it is 85%.

Since a beehive represents a defined area with known flow rates of the incoming and outflowing air and only minor variations of the internal temperature, a dispenser described above can advantageously be used for controlled release of formic acid to treat Varroa. With a suitable choice of the concentration of aqueous formic acid, treatment of parasitic mites is effective without burdening the colony too much.

For example, it may be advantageous to gradually reduce or increase the dispensing rate before a certain desired dispensing rate is achieved, which is then maintained within a preferred tolerance. This is advantageous for example for the described use of formic acid in the treatment of Varroa.

It is an additional advantage of the exemplary embodiments described, that the risk of injury is reliably prevented by the exclusion of direct contact with the liquid ingredients to the environment, people, animals and plants. This advantage is particularly obvious in the case of formic acid used for varroa control.

Another advantage of the described embodiments or their use for the treatment of animal and plant diseases by the administration of volatile substances and active ingredients in the air breathed is to gradually increase the partial pressure of each substance to a desired value which is then maintained

This is advantageous for example in the case of Varroa treatment in beehives, since the partial pressure of the acid in the hive thus gradually increases before it reaches its effective value. This method allows the bees to get used to the treatment and causes less loss in the colony.

In contrast to previously known dispensers which require pumps, heaters, wicks or other additional means for the dispensing of volatile substances, the proposed use of liquid mixtures of an active ingredient with an auxiliary liquid which can form an azeotrope is much easier. Another advantage is that the composition of liquid mixtures and mixtures of the gas phase can be changed during dispensing operation. With prior art dispensing devices even then this is not possible when used with such a mixture that can form an azeotrope.

One advantage of the release of organic acids, or other liquids which are aggressive in a concentrated form, with the present invention is that the liquid form of volatile substance at the membrane is not in direct contact with the environment but only gas or steam is released, what is in contrast to usual evaporation surfaces.

The use of known dispensers is mostly limited to the use of a fixed composition of the liquid volatile substance in the container, which does not change during the application.

In contrast to this, here the selected fluid mixture to be filled in the dispenser represents a selected azeotrope-forming mixture of substances, namely a liquid mixture of volatile active substances and a custom auxiliary liquid, selected to provide the desired release profile and to fit the respective application. The systematic change in the composition of this mixture in interaction with the concentration in the gas phase of the environment is used to control the kinetics of release of volatile substance in the ambient air of a delimited or enclosed space. For example, the selective modification of the composition of this mixture in interaction with the concentration in the gas phase of the environment is used to the achieve a release profile having two phases.

The present invention has been explained with reference to exemplary embodiments. These embodiments should not be limiting to the present invention. The following claims are a first, non-binding attempt to define the invention in general.

1. Device for dispensing a volatile active substance, comprising a container (3) which is at least partially restricted by a transpiration layer (2), whereby
the container (3) is at least partially filled by a liquid mixture of at least one volatile liquid substance and an auxiliary liquid,
the liquid mixture of liquid active substance and auxiliary liquid is capable to form an azeotrope, and
the transpiration layer (2) is impermeable to the liquid pahse of the liquid mixture, but permeable to its gaseous phase.

2. Device according to claim 1, wherein the volatile active substance is selected from the group comprising formic acid, a pheromone or a substance that is acting on insects like a pheromone, a hormone or a substance with hormonal effects on plants or animals, a fungicide, an acaricide, an insecticide, a scent, a fragrance and a biological active substance.

3. Device according to claim 1, wherein the transpiration layer (2) comprises an oleophobic polymer or a polymer treated to be oleophobic or an oleophobic paper or a paper treated to be oleophobic.

4. Device according to claim 1, further comprising a removable impermeable barrier layer (1), which covers the transpiration layer (2) to prevent evaporation of the liquid mixture.

5. Device according to claim 4, wherein the barrier layer (1) comprises at least one linear breaking point and/or a zone of reduced thickness which is applicable for the removal of a part and/or section of the barrier layer (1), so that after the partial or complete removal of the barrier layer (1) at least a portion of transpiration layer (2) is exposed.

6. The device according to claim 1, wherein the concentration of the active substance in the liquid mixture is higher than the concentration of the active substance at the azeotropic point of the liquid mixture.

7. The device according to claim 1, wherein the concentration of the active substance in the liquid mixture is below the concentration of the active substance at the azeotropic point of the liquid mixture.

8. Device according to claim 1, wherein the container is formed by a receptacle having at least one opening, the transpiration layer (2) closes the opening.

9. Method for manufacturing a device for dispensing a volatile active substance, comprising:
providing a container;
at least partially filling the container with a liquid mixture comprising at least one liquid volatile substance and an auxiliary liquid, wherein the active substance and the auxiliary liquid are selected such that the liquid mixture of the liquid active substance and the auxiliary liquid is capable to form an azeotrope;
sealing the container with a transpiration layer (2), that is impermeable for the liquid phase of the mixture and permeable to its gaseous phase.

10. The method of claim 9, further comprising:
Application of a removable barrier layer on the transpiration layer (2).

11. Method for manufacturing a device for dispensing a volatile active substance, comprising:
Providing a container that is at least partially restricted by a transpiration layer (2);
at least partially filling the container with a liquid mixture comprising at least one liquid volatile substance and an auxiliary liquid, wherein the active substance and the auxiliary liquid are selected such that the liquid mixture of the liquid active substance and the auxiliary liquid is capable to form an azeotrope;
where the transpiration layer (2) is impermeable to the liquid phase of the liquid mixture but permeable to its gaseous phase.

12. Using the device of claim 1 for the following applications:
Controlling parasitic mites in beehives;
Controlling pests in a storage room and/or storage space;
Deodorization of interior air, such as vehicle interiors, airplane interiors, showrooms, living rooms, bedrooms and toilet facilities;
Relief from respiratory ailments.
